(19) United States
(12) Patent Application Publication
(10) Pub. No.: US 2023/0255613 A1
(43) Pub. Date: Aug. 17, 2023

(54) SUTURE ANCHORS WITH SPECIALIZED CORTICAL AND CANCELLOUS BONE-ENGAGING SEGMENTS HAVING OPTIMIZED THREAD PATTERNS

(71) Applicant: HS WEST INVESTMENTS, LLC, Sandy, UT (US)

(72) Inventor: Hugh S. WEST, JR., Sandy, UT (US)

(21) Appl. No.: 18/108,827

(22) Filed: Feb. 13, 2023

Related U.S. Application Data
(60) Provisional application No. 63/310,693, filed on Feb. 16, 2022.

(51) Int. Cl. A61B 17/04

(52) U.S. Cl. CPC A61B 17/0401; A61B 2017/0414; A61B 2017/0441

(57) ABSTRACT

Suture anchors having specialized cortical and cancellous bone-engaging segments with optimized thread patterns to enhance mechanical fixation to cortical and cancellous bone tissue. This is accomplished by providing multiple (e.g., two) starts of fine helical threads in the cortical bone-engaging segment and coarse helical threads with fewer starts (e.g., one start), and greater spacing, height, and depth in the cancellous bone-engaging segment. The threaded anchor body includes a central bore, with a socket at a proximal opening and a suture-attachment structural member therein. The bore only extends through the cortical bone-engaging segment but not the cancellous bone-engaging segment. This permits the cancellous bone-engaging segment to have a substantially reduced root diameter, which substantially increases the height and depth, and therefore the mechanical bite, of the cancellous bone-engaging threads.

### CROSS REFERENCE TO RELATED APPLICATION

This application claims the benefit of U.S. Provisional Application No. 63/310,693, filed Feb. 16, 2022, which is incorporated herein by reference in its entirety.

### BACKGROUND

### 1. The Field of the Invention

The present invention relates to suture anchors with specialized cortical and cancellous bone-engaging segments, each having an optimized thread pattern for engaging and being affixed within, respectively, cortical and cancellous bone tissue.

### 2. Related Technology

Soft tissue injuries can occur from repetitive stress and/or acute trauma. Such injuries may require surgery to correctly reattach the soft tissue to the bone to promote proper healing. Various devices have been used to reattach soft tissue to bone, including staples, wedges, plugs, screws, and sutures alone. Threaded suture anchors, such as bone screws, can provide a secure location in bone where a suture can be anchored to the bone. In these systems, a suture is tied between the suture anchor and soft tissue. Providing a secure attachment point for attaching the suture is important for long-term healing to offset pullout and other forces that can compromise the repair.

Despite advances in suture anchors, soft tissue repair surgeries can fail, either because the suture anchor becomes dislodged or the suture tears through the soft tissue. As force is applied to the suture, the suture can cut through the soft tissue like a cheese wire, leaving soft tissue detached from the bone. If one suture fails, this can place more stress on other sutures, increasing the likelihood that they will fail in like manner.

Using a greater number of sutures per unit area of soft tissue can minimize suture attachment failure. However, the number of sutures is typically limited by the space available for inserting suture anchors. Although additional sutures can be connected to a single suture anchor, double and triple loading of anchors increases the forces applied to the suture anchor and increases the likelihood that the anchor will fail by being pulled out of the bone into which it is secured.

Suture anchors can fail for various reasons. Some suture anchors are not fully threaded to the proximal end of the anchor, where the anchor is adjacent to hard cortical bone. In such anchors, the proximal end is not threaded because the driver tool used to insert the suture anchor into bone includes a socket that fits over and engages a hexagonal protrusion (or drive head). Because it would cause patient discomfort and potential tissue damage for the hexagonal drive head to extend above the bone surface, such anchors must be driven into the bone until the drive head is at or below the bone surface. Since the drive head is not threaded, the anchor does not engage cortical bone near the surface, but only soft cancellous bone beneath the cortical bone layer. Such suture anchors are incapable of engaging cortical bone, which is the strongest and best bone to reliably engage.

Some suture anchors are fully threaded to the proximal end and are therefore able, at least in theory, to engage both cancellous and cortical bone. Examples are disclosed in U.S. Pat. Nos. 8,343,186, 8,623,052, 8,801,755, 9,622,739, and 10,537,319 to Dreyfuss et al. Such anchors are characterized as having an internal driver socket and suture pin in a proximal threaded half and a single continuous thread extending along nearly the entire length of the anchor body, with essentially the same root diameter at the proximal and distal ends of the thread.

Other examples are disclosed in U.S. Pat. Nos. 9,521,999 and 10,595,842 to Dreyfuss et al. Such anchors are characterized as having an internal driver socket and internal suture loop near a proximal end, a constriction near a distal end for a suture knot, and a single continuous thread extending along the entire length of the anchor body. The root diameter appears to be somewhat larger at the proximal end than at the distal end of the thread.

Yet other examples of useful suture anchors are disclosed in U.S. Pat. Nos. 7,322,978, 8,114,127, 9,161,748, 9,402,618, 9,820,732, and 11,090,035 to West, Jr. Such anchors are characterized as having an internal bore passing through an entirety of the threaded anchor body region, a pair of suture attachment pins in a distal threaded half of the anchor body, and a continuous thread that extends between the proximal and distal threaded regions. In one embodiment, a second thread is positioned in the proximal threaded region to enhance engagement with cortical bone. An important feature of the West patents is the placement of the suture attachment pins deep within the internal bore in the distal threaded half of the anchor body to reduce loosening or withdrawal of the anchor from the bone via torquing forces.

A problem remains, however, that when suture anchors are made very small (e.g., having a major diameter of less than 5 mm, 4.5 mm, 4 mm, 3.5 mm, or 3 mm), such as where it is desired to place multiple anchors in a small location, such as in shoulder repairs, the thread patterns in prior art anchors are too undifferentiated to provide optimal engagement of and fixation to, respectively, cortical and cancellous bone. Rather, at very small scales, threads designed to engage cancellous bone are virtually indistinguishable from threads designed to engage cortical bone.

Accordingly, there remains a need to provide suture anchors that provide reliable and secure engagement of and fixation to both cortical and cancellous bone tissues.

### SUMMARY

Disclosed herein are suture anchors having specialized cortical and cancellous bone-engaging segments with optimized thread patterns that greatly enhance fixation of the anchor to, respectively, cortical and cancellous bone tissue. This is accomplished by providing multiple (e.g., two) starts of fine helical threads in the cortical bone-engaging segment and coarse helical threads with fewer starts (e.g., one start), and greater spacing, height, and depth in the cancellous bone-engaging segment.

To further achieve this result, the threaded anchor body includes a central bore, which provides a socket at a proximal opening and a rigid suture-attachment member at or near a distal end of the central bore, which only extends through the cortical bone-engaging segment but does not pass into the cancellous bone-engaging segment. This permits the root diameter of the cancellous bone-engaging segment to be substantially reduced without sacrificing strength, which substantially increases the height and depth, and therefore the mechanical bite (or purchase), of the cancellous bone-engaging threads to cancellous bone. In some cases, the root diameter of the cancellous bone-engaging segment can be only slightly greater than, equal to, or even less than, the diameter of the central bore in the cortical bone-engaging segment. This substantially increases the ratio of thread height in the cancellous bone-engaging segment to thread height in the cortical bone-engaging segment.

Bones typically have a hard outer layer of bone tissue, which is cortical bone, and a relatively soft and porous interior region beneath the cortical bone, which is cancellous bone. The cortical bone provides mechanical strength and hardness while cancellous bone provides bulk, flexibility, and greater ability to transport nutrients through the bone tissue.

The relative hardness of cortical bone tissue compared to the relative softness of cancellous bone tissue means that a single thread pattern is suboptimal and inadequate to optimize the ability of a threaded suture anchor to be screwed into and remain anchored within, respectively, the cortical and cancellous bone regions. Threads that are too fine (excessively small thread height) more easily strip and/or pull out from cancellous bone tissue, while threads that are too coarse (excessively large thread height) and spaced-apart are more difficult to drive into hard cortical bone and lack a sufficient number of thread-turns per unit of axial length to reliably engage and remain embedded within a thin cortical bone layer.

The suture anchors disclosed herein are configured with differential thread patterns and root diameters that: (1) maximize the ability of even tiny anchors (e.g., having major diameters less than 5 mm, 4.5 mm, 4 mm, 3.5 mm, or 3 mm) to obtain reliable purchase (or mechanical engagement) with, longitudinally screw into, and remain embedded within soft cancellous bone and (2) maximize the ability of the anchors to be screwed into and reliably engage hard cortical bone. In addition, the differential thread patterns and root diameters can also greatly enhance the bone-engaging ability of larger anchors (e.g., having major diameters greater than 5 mm, 5.5 mm, 6 mm, 6.5 mm, or 7 mm).

In a preferred but non-limiting embodiment, the cancellous bone-engaging segment of the suture anchor includes a single helical thread or “start” with sufficient pitch, i.e., spacing between adjacent thread turns, to provide a desired “lead”, which is the distance along the anchor's axis that is covered by one complete rotation of the anchor (360°). In contrast, the cortical bone-engaging segment includes two or more parallel helical threads or starts, each having the same pitch as the helical thread of the cancellous bone-engaging segment. By maintaining the same pitch, a “main” helical thread or start in the cortical bone-engaging segment is essentially a continuation of the helical thread in the cancellous bone-engaging segment and can follow the impression created by the cancellous bone-engaging thread as the bone anchor is driven longitudinally into the bone. The one or more additional parallel helical threads or starts of the cortical bone-engaging segment are positioned between the main helical thread or start and, being significantly finer than the coarse helical threads of the cancellous bone-engaging segment, are able to readily form a new, but shallower, impression in the cortical bone layer in order to maximize engagement and fixation of the anchor within the relatively thin cortical bone layer.

To better differentiate the respective thread patterns in the cortical and cancellous bone-engaging segments and enable the threaded anchor body to reliably engage and become embedded within, respectively, cortical and cancellous bone tissues, the suture anchors disclosed herein can have optimized ratios of major diameter, first (or cortical) root diameter of the cortical bone-engaging segment, second (or cancellous) root diameter of the cancellous bone-engaging segment, and differential thread heights in the cortical and cancellous bone-engaging segments.

The “major diameter” is the diameter spanning the outer edges of the helical threads on opposite sides of the anchor body in the cortical and cancellous bone-engaging segments. The “root diameter” is the diameter of the anchor body spanning the troughs between the raised threads. The “thread height” is the distance between the outer thread edges and the adjacent root surface. While the major diameter can be substantially constant along the length of the suture anchor, the root diameter is significantly greater in the cortical bone-engaging segment than in the cancellous bone-engaging segment, and the thread height is significantly greater in the cancellous bone-engaging segment than in the cortical bone-engaging segment.

The “first (or cortical) major diameter” is the diameter spanning the outer edges of the fine cortical threads on opposite sides of the anchor body in the cortical bone-engaging segment. The “first (or cortical) root diameter” is the diameter of the anchor body spanning the troughs between the raised threads in the cortical bone-engaging segment. The “first (or cortical) thread height” is the distance between an outer root surface of the anchor body in the cortical bone-engaging segment and outer edges of the fine cortical threads. The cortical thread height can also be defined as the difference between the cortical major diameter and the cortical root diameter, divided by two (assuming symmetrical or uniform threads). In some embodiments, the cortical major diameter, cortical root diameter, and cortical thread height can be substantially constant. In other embodiments, they can vary (e.g., taper in the proximal to distal direction).

The “second (or cancellous) major diameter” is the diameter spanning the outer edges of the coarse cancellous threads on opposite sides of the anchor body in the cancellous bone-engaging segment. The “second (or cancellous) root diameter” is the diameter of the anchor body spanning the troughs between the raised threads in the cancellous bone-engaging segment. The “second (or cancellous) thread height” is the distance between an outer root surface of the anchor body in the cancellous bone-engaging segment and outer edges of the coarse cancellous threads. The cancellous thread height can also be defined as the difference between the cancellous major diameter and the cancellous root diameter, divided by two (assuming symmetrical or uniform threads). In some embodiments, the cancellous major diameter, cancellous root diameter, and cancellous thread height can be substantially constant. In other embodiments, they can vary (e.g., taper in the proximal to distal direction).

The ratio of the major diameter to the first (or cortical) root diameter can be greater than 1.15:1, such as greater than about 1.2:1, 1.22:1, or 1.24:1, and less than about 1.5:1, 1.45:1, 1.4:1, 1.35:1, or 1.3:1. The ratio of the major diameter to the second (or cancellous) root diameter can be greater than 1.7:1, such as greater than about 1.8:1, 1.9:2:1, or 2.5:1, and less than about 4:1, 3.5:1, or 3:1. The ratio of the first (or cortical) root diameter to the second (or cancellous) root diameter can be greater than 1.5, such as greater than about 1.6:1, 1.8:2, 2:1, 2.2:1, or 2.5:1, and less than about 3.5:1, 3.25:1, 3:1, or 2.75:1. The ratio of the thread height of the coarse cancellous threads in the cancellous bone-engaging segment to the thread height of the fine cortical threads in the cortical bone-engaging segment can be greater than 1.5:1, such as greater than about 1.6:1, 1.7:1, 1.8:1, or 1.9:1, and less than 4:1, such as less than about 3.8:1, 3.6:1, 3.4:1, 3.2:1 or 3:1.

As stated above, the central bore only extends at least partially though the cortical bone-engaging segment such that the rigid suture-attachment member is positioned within the cortical bone-engaging segment and not the cancellous bone-engaging segment. In this way, the anchor body is solid rather than hollow in the cancellous bone-engaging segment. This permits the root diameter in the cancellous bone-engaging segment to be only slightly greater than, equal to, or even less than the diameter of the central bore. This permits the anchor body to have a substantially smaller root diameter in the cancellous bone-engaging segment than if the central bore were to extend into, such as through, the cancellous bone-engaging segment, while having the same or greater strength.

While positioning the rigid suture-attachment member in the cortical bone-engaging segment may arguably increase lateral torque and pull-out forces at the proximal end of the anchor compared to suture anchors where the rigid suture-attachment member is positioned within the cancellous bone-engaging segment, it has now been found that such forces are more than offset by the increased stabilization forces provided by the substantially coarser cancellous threads. The net result is a suture anchor that can be made significantly smaller yet obtain adequate and even superior purchase and stability within cortical and cancellous bone regions compared to prior art suture anchors that lack the more exaggerated differences between the cortical and cancellous bone-engaging threads as disclosed herein.

Even when making larger suture anchors (e.g., having a major diameter greater than 5 mm, 5.5 mm, 6 mm, 6.5 mm, or 7 mm), the exaggerated differences between the fine cortical and coarse cancellous bone-engaging threads as disclosed herein (e.g., conforming to the ratios of the major diameter, cortical root diameter, cancellous root diameter, and differential thread heights, as discussed above) yield a suture anchor having superior strength and pull-out resistance as a result of eliminating the central bore from, and reducing the root diameter of, the cancellous bone-engaging segment. Without the central bore passing therethrough, the cancellous bone-engaging segment can have a substantially smaller root diameter, which greatly increases thread coarseness and cancellous bone purchase, while having the same or greater strength.

Optimizing the thread patterns for engaging, respectively, hard cortical bone and soft cancellous bone allows the suture anchor to more securely engage both types of bone tissue. This permits the suture anchor to be loaded with a plurality of sutures if desired without undermining the stability of the surgical repair. Each additional suture per anchor reduces stresses exerted by each individual suture tied to soft tissue, which helps prevent the sutures from cutting through the soft tissue like a wire through cheese.

Apart from the foregoing thread patterns and definitions, the central bore that extends at least partially through the cortical bone-engaging segment can have the shape of a female socket configured to receive therein a corresponding-shaped male driver tip. Example shapes include, but are not limited to, hexagonal, pentagonal, square, triangular, star-shaped, oval, or other non-circular or non-cylindrical geometric shape.

A pin or other rigid member disposed across the central bore of the cortical bone-engaging segment provides an attachment mechanism for attaching (e.g., looping) a suture thereto. The pin or other rigid member can be made of the same or different material as the anchor body. Non-limiting examples of materials for fabricating the anchor body and rigid member include stainless steel, titanium, nickel-titanium alloy, other metals or alloys, and polymers (e.g., polylactic acid, polyether ether ketone (PEEK), nylon, polyester, polyethylene, polypropylene, and the like).

Although the suture anchor can be fully threaded, in some embodiments the anchor body may optionally include a non-threaded tip portion or extension at the distal end. The non-threaded tip portion or extension can provide additional resistance to torquing forces and tipping. The distal tip of suture anchor tip may be configured to be self-tapping and/or self-drilling, such as by providing a cutout on a side near the tip and/or providing a taper at the distal end.

These and other advantages and features of the invention will become more fully apparent from the following description and appended claims, or may be learned by the practice of the invention as set forth hereinafter.

### BRIEF DESCRIPTION OF THE DRAWINGS

In order to describe the manner in which at least some of the advantages and features of the invention may be obtained, a more particular description of embodiments of the invention will be rendered by reference to specific embodiments thereof which are illustrated in the appended drawings. Understanding that these drawings depict only typical embodiments of the invention and are not therefore to be considered to be limiting of its scope, embodiments of the invention will be described and explained with additional specificity and detail through the use of the accompanying drawings, in which:

FIGS. 1A-1C are perspective and cross-sectional views that illustrate a first embodiment of a suture anchor having specialized cortical and cancellous bone-engaging segments;

FIGS. 2A-2C are perspective and cross-sectional views that illustrate a second embodiment of a suture anchor having specialized cortical and cancellous bone-engaging segments;

FIGS. 3A-3C are perspective and cross-sectional views that illustrate a third embodiment of a suture anchor having specialized cortical and cancellous bone-engaging segments;

FIGS. 4A-4C are perspective and cross-sectional views that illustrate a fourth embodiment of a suture anchor having specialized cortical and cancellous bone-engaging segments;

FIGS. 5A-5C are exploded and assembled views, respectively, of a suture anchor system that includes a suture anchor with associated suture(s) and a driver used to install the suture anchor into a bone;

FIG. 6 is a cross-sectional view that illustrates a suture anchor having specialized cortical and cancellous bone-engaging segments with associated sutures installed in a bone having distinct cortical bone and cancellous bone regions;

FIG. 7 illustrates an exemplary suture anchor with associated sutures installed in a humerus bone, with sutures anchored to the bone and securing a rotator cuff to the bone; and

FIG. 8 illustrates exemplary suture anchors with associated sutures installed in a hip socket with sutures anchored to the bone and securing a labrum to the bone.

### DETAILED DESCRIPTION

The present disclosure relates to suture anchors used to attach one or more sutures to a bone during a surgical procedure, such as a shoulder or hip repair surgery, where it is desired to provide secure and reliable fixation to the bone. The suture anchors include specially designed cortical and cancellous bone-engaging segments having optimized thread patterns that enhance fixation of the anchor to cortical bone and cancellous bone respectively.

The cancellous bone-engaging segment has a smaller root diameter and is provided with coarse helical threads having fewer starts (e.g., one start), and greater spacing, height, and depth compared to the fine helical threads in the cortical bone-engaging segment, which has a larger root diameter and is provided with multiple (e.g., two) starts of fine helical threads having smaller height and depth than the course threads, with each start preferably having the same pitch as the start of the coarse helical thread(s). The coarse helical threads in the cancellous bone-engaging segment, which extend at least partially to the distal tip of the suture anchor, are inserted (i.e., screwed) into the bone first, passing through the relatively thin cortical bone layer and into the thicker, but softer and more porous, cancellous bone region, where they are able to reliably gain mechanical purchase and advance into the cancellous bone without stripping the bone. The fine helical threads of the cortical bone-engaging segment trail the coarse helical threads into the bone, where they, in combination with the larger root diameter, engage the harder and stronger layer of cortical bone. Because the fine helical threads are closer together, they provide greater fixation to cortical bone compared to coarser threads. In addition, the larger root diameter in the cortical bone-engaging segment further compresses against the cortical bone to promote even greater fixation of the fine helical threads to cortical bone.

To increase the differential in root diameters between the cortical bone-engaging segment and the cancellous bone-engaging segment, the threaded anchor body includes a central bore (containing a socket and rigid suture-attachment member) that only extends through the cortical bone-engaging segment but not the cancellous bone-engaging segment. The absence of a central bore in the cancellous bone-engaging segment permits the root diameter of the cancellous bone-engaging segment to be substantially smaller than the root diameter of the cortical bone-engaging segment. Because the cancellous bone-engaging segment is solid rather than hollow, its root diameter can be reduced substantially without compromising strength and rigidity. The smaller root diameter, in turn, allows for coarse helical threads having substantially greater height and depth in order gain better mechanical purchase with soft cancellous bone compared to anchors having a central bore extending through most or all of the anchor body. The root diameter of the cancellous bone-engaging segment can be slightly greater than, equal to, or less than, the diameter of the central bore in the cortical bone-engaging segment. This substantially increases the ratio of thread height in the cancellous bone-engaging segment to thread height in the cortical bone-engaging segment.

The relative hardness of cortical bone tissue compared to the relative softness of cancellous bone tissue means that a single thread pattern is suboptimal and inadequate to optimize the ability of a threaded suture anchor to be screwed into and remain anchored within, respectively, cortical and cancellous bone. Threads that are too fine (e.g., that have excessively small thread height) more easily strip and/or pull out from cancellous bone tissue, while threads that are too coarse (e.g., that have excessively large thread height) and spaced-apart are more difficult to drive into hard cortical bone and lack a sufficient number of thread turns per unit of axial length to reliably engage and remain embedded within a thin cortical bone layer. Bone anchors having a central bore passing through both the cortical and cancellous bone-engaging segments cannot be fully optimized to have specialized cortical and cancellous bone-engaging threads.

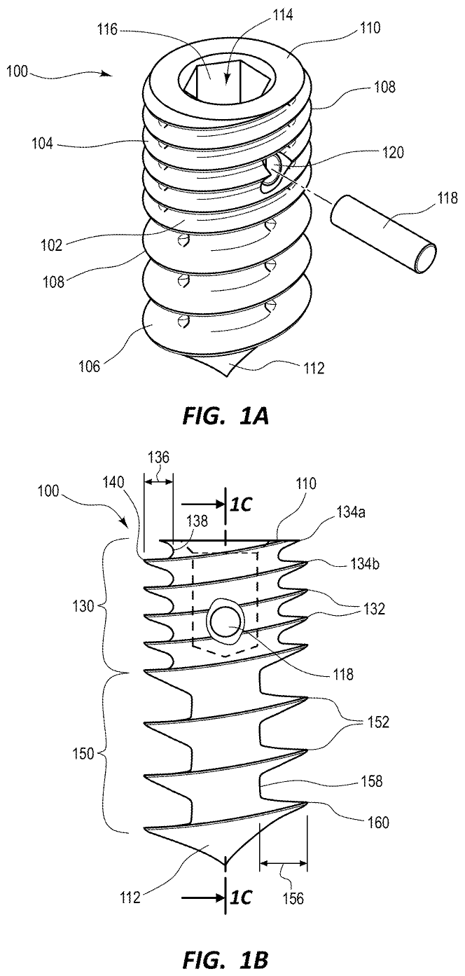

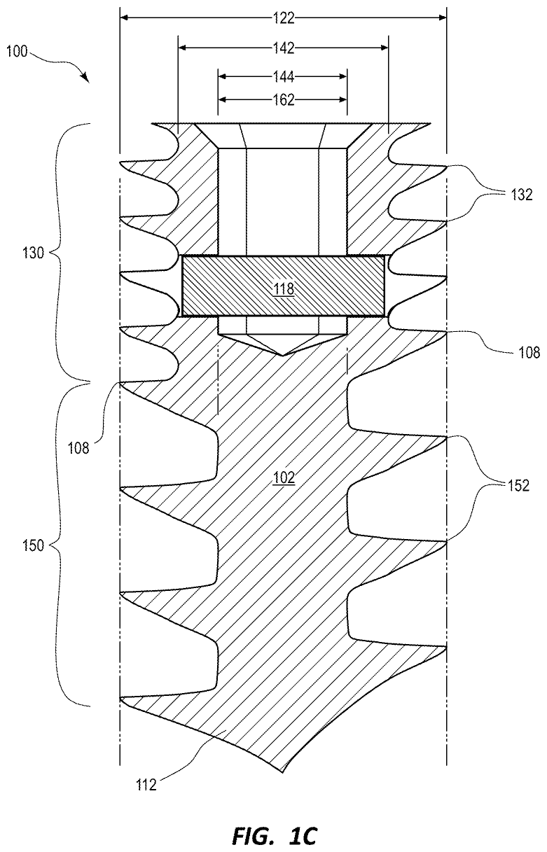

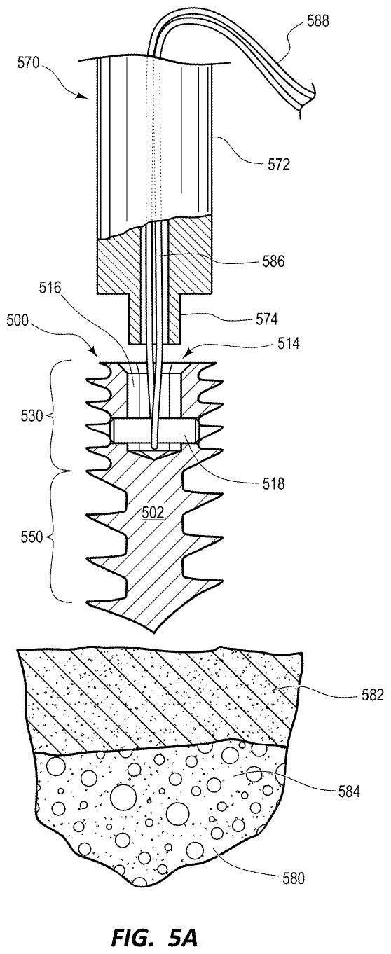

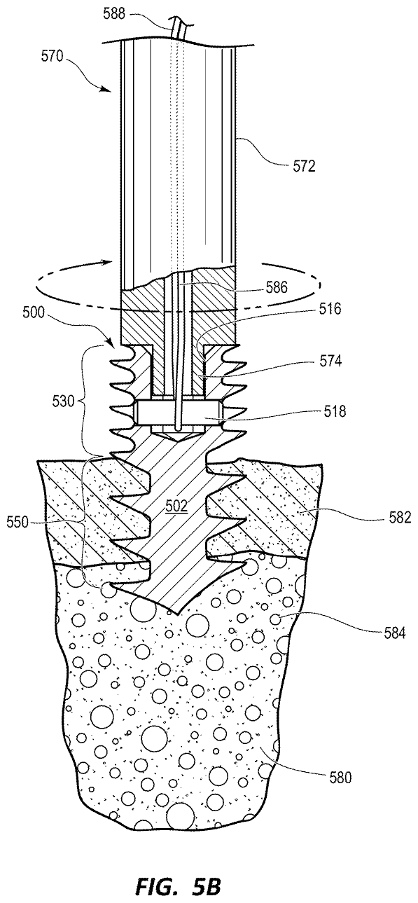

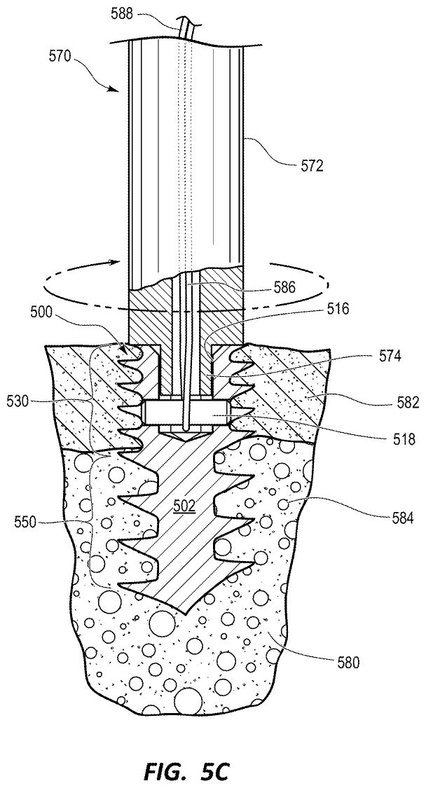

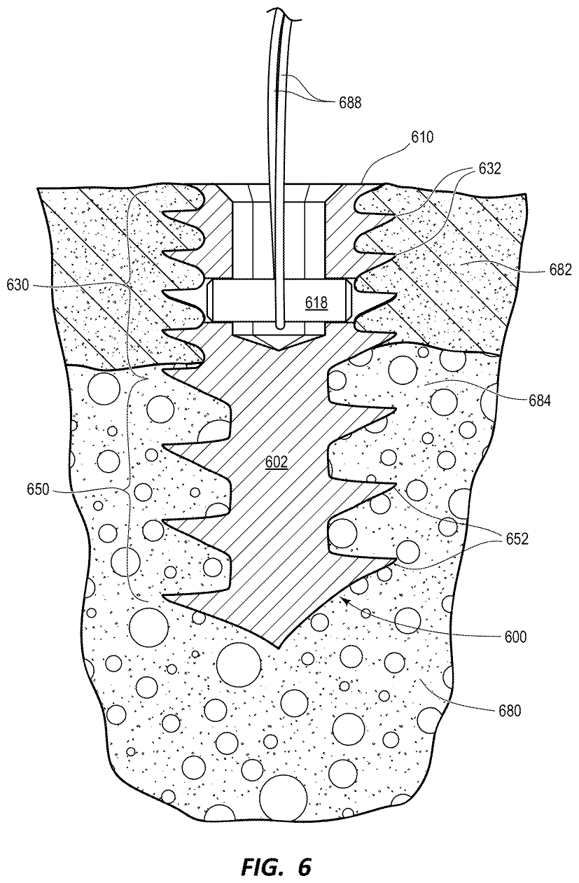

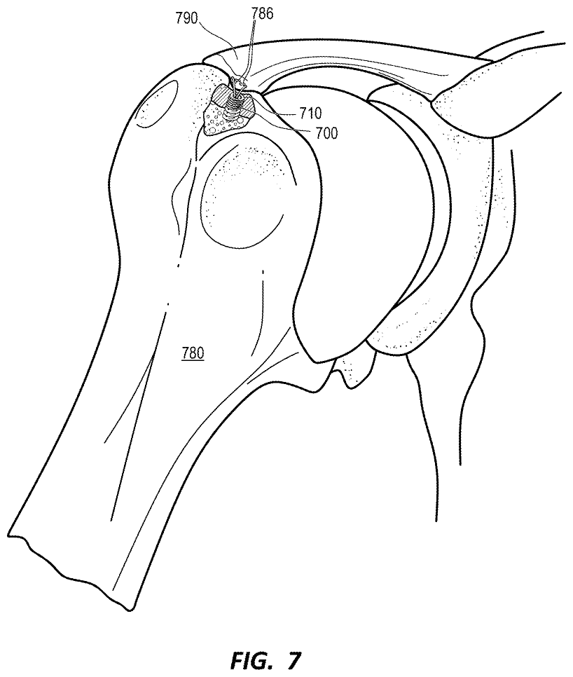

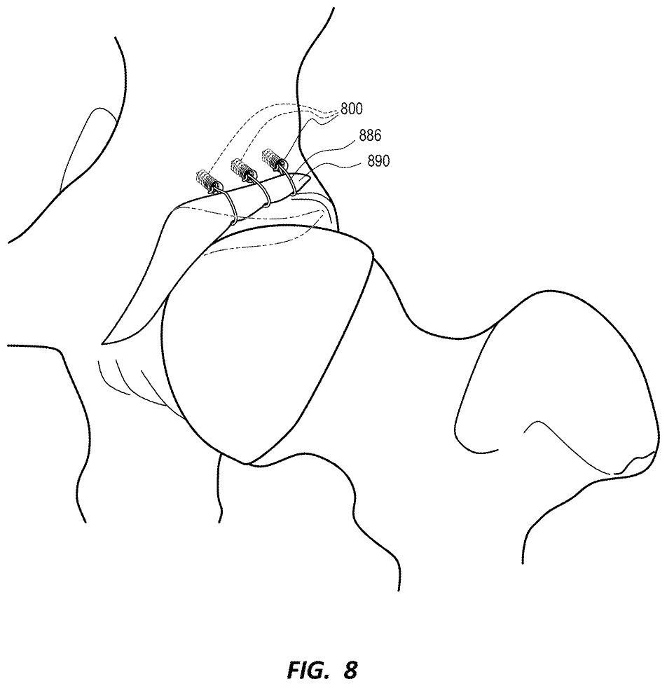

Turning now to the drawings, FIGS. 1-4 schematically illustrate example embodiments of suture anchors having specialized cortical and cancellous bone-engaging segments that optimize engagement with and fixation to, respectively, cortical and cancellous bone regions at a surgical site. FIGS. 5A-5C schematically illustrate the implantation of a suture anchor having an associated suture into a bone using a driver. FIG. 6 schematically illustrates a suture anchor in which the cortical bone-engaging segment is embedded within an outer cortical bone layer and the cancellous bone-engaging segment is embedded within the underlying cancellous bone region. The suture anchors can be used in any desired manner and for any desired surgical procedure. In some implementations, they are particularly well-suited for rotator cuff shoulder repair surgeries. In other implementations, they can be used in labral hip repair surgeries. FIGS. 7 and 8 schematically illustrate suture anchors used in shoulder and hip surgeries, respectively.

FIGS. 1A-1C more particularly illustrate perspective and cross-sectional views of a first embodiment of a suture anchor 100 according to the disclosure having an anchor body 102 extending along a longitudinal axis between a proximal end 104 and a distal end 106. FIG. 1C is a cross-sectional view taken along line 1C-1C. The anchor body 102 can have a length ranging from about 6 mm to about 20 mm, such as about 7 mm to about 15 mm, and with an appropriate major diameter to have a desired aspect ratio. The anchor body 102 can be made of any appropriate material, examples of which include, but are not limited to, stainless steel, titanium, nickel-titanium alloy, other metals, other alloys, and polymers, such as polylactic acid, polyether-ether ketone (PEEK), nylon, polyester, polyethylene, polypropylene, and the like.

Helical threads 108 are disposed on an outer surface of the anchor body 102 and extend between a proximal face 110 and a distal tip 112. The outer edges of the helical threads 108 define a major diameter 122 of the anchor body 102 (FIG. 1C). The major diameter 122 is illustrated as being substantially constant along the length of the anchor body 102, although it can be made to vary if desired, such having a taper (e.g., toward the distal tip 112). Although FIGS. 1A-1C show the suture anchor 100 being fully threaded between the proximal face 110 and distal tip 112, it is within the scope of the disclosure to include a non-threaded portion or extension, which may be included to guide the anchor body 102 into a preformed (e.g., pre-drilled) pilot hole and/or to provide additional length that can increase resistance to tipping forces applied by lateral-pulling sutures (see FIG. 7).

The anchor body 102 further includes a central bore 114 that opens at the proximal face 110 and includes a socket 116 (illustrated as hexagonal) for receiving therein a correspondingly-shaped driver tip (see, e.g., FIGS. 5A-5B). The socket can have any other desired shape that permits engagement with a correspondingly shaped driver tip, such as pentagonal, square, triangular, star-shaped, oval, or other non-circular or non-cylindrical geometric shape.

The suture anchor 100 includes a rigid member 118 (e.g., a pin) disposed in and at or near the bottom of the central bore 114, which provides an attachment site for attaching one or more sutures thereto (see, e.g., FIGS. 5-6). The rigid member 118 can be inserted through a hole 120 through one or both opposing sidewalls of the anchor body 102 (FIG. 1A) so as to extend at least partially across the central bore 114 (FIGS. 1B-1C). The rigid member 118 can be substantially straight as shown, or it may be curved, bent, or have variable diameter to provide a desired function or manufacturing capability. The rigid member 118 can be made from the same or different material used to make the anchor body 102 (such materials being discussed above). As illustrated, the rigid member 118 is positioned at a bottom of the central bore 114, which maximizes the depth of socket 116 for receiving a driver tip. The rigid member 118 can be made from a radiopaque material so that placement of sutures can be inspected and inferred by X-ray following surgery. Any portion of the anchor body 102 can be radiopaque if desired, as can any suture(s) attached thereto.

The anchor body 102 further includes highly differentiated thread patterns that optimize engagement with and fixation to, respectively, cortical and cancellous bone regions at the implant site. More particularly, the anchor body 102 includes a cortical bone-engaging segment 130 with fine cortical threads and a cancellous bone-engaging segment 150 with coarse cancellous threads.

With particular reference to FIGS. 1B-1C, the cortical bone-engaging segment 130 includes fine helical threads 132 having multiple starts 134 (e.g., two starts 134a and 134b) having the same pitch and being parallel to each other. As illustrated in FIG. 1B, the fine helical threads 132 have a thread height 136 defined by a difference between an outer root surface 138 of the anchor body 102 and outer edges 140 of the fine helical threads 132.

As seen in FIG. 1C, the cortical bone-engaging segment 130 has a first (or cortical) root diameter 142. The central bore 114 in the cortical bone-engaging segment 130 has a bore diameter 144 spanning a distance between opposing sidewalls or angles where adjacent socket faces intersect, the greatest of which is the maximum bore diameter. The bore diameter 144 can therefore differ depending on the shape of the socket and where measured. The rigid member 118 is illustrated as being embedded in opposing sidewalls of the anchor body 102 at or near the bottom of and extending across the central bore 114. It should be understood that the rigid member 118 may only be attached to one sidewall and extend only partially across the central bore 114.

As further shown in FIGS. 1B-1C, the cancellous bone-engaging segment 150 includes coarse helical threads 152 having a single start, and preferably having the same pitch as each start 134 of the fine helical threads 132 in the cortical bone-engaging segment 130. As illustrated in FIG. 1B, the single start of coarse helical threads 152 is a continuation of one of the starts 134 (i.e., start 134a) of the fine helical threads 132. By maintaining the same pitch between the coarse helical threads 152 and each start 134 of the fine helical threads 132, a “main” fine helical thread (i.e., start 134a) in the cortical bone-engaging segment 130 can follow the impression cut by the single start of coarse helical threads 152 as the bone anchor 100 is driven longitudinally into the bone. The secondary start 134b does not follow the impression cut by the single start of coarse helical threads 152 as the bone anchor 100 is driven longitudinally into the bone but creates a new impression between the initial impression cut by the coarse helical threads 152. This further assists in engaging hard cortical bone. The cancellous bone-engaging segment 150 has a second (or cancellous) root diameter 162 (FIG. 1C).

As further illustrated in FIG. 1B, the coarse helical threads 152 have a thread height 156 defined by a difference between an outer root surface 158 of the anchor body 102 and outer edges 160 of the coarse helical threads 152. As further illustrated, the thread height 156 of the coarse helical threads 152 is greater than the thread height 136 of the fine helical threads 132. In preferred embodiments, the ratio of the thread height 156 of the coarse helical threads 152 to the thread height 136 of the fine helical threads 132 can be greater than 1.5:1, such as greater than about 1.6:1, 1.7:1, 1.8:1, or 1.9:1, and less than 4:1, such as less than about 3.8:1, 3.6:1, 3.4:1, 3.2:1 or 3:1.

As schematically illustrated in FIG. 1C, the major diameter 122 of the anchor body 102 is the distance between opposing outer edges of the helical threads 108 on opposite sides of the anchor body 102 (e.g., the diameter of an imaginary cylinder around the outer edges of the helical threads 108). The first (or cortical) root diameter 142 is the distance between opposing root surfaces 138 in the troughs between adjacent fine helical threads 132 on opposite sides of the anchor body 102 in the cortical bone-engaging segment 130. The second (or cancellous) root diameter 162 is the distance between opposing outer root surfaces 158 in the troughs between adjacent fine helical threads 152 on opposite sides of the anchor body 102 in the cancellous bone-engaging segment 150.

As illustrated, the major diameter 122, the first root diameter 142, and the second root diameter 162 are substantially constant. It should be understood, however, that in other embodiments, one or more of them can vary (i.e., taper toward the distal tip). As further illustrated, the major diameter 122 is greater than both the first root diameter 142 and the second root diameter 162, the first root diameter 142 is greater than the second root diameter 162, and the second root diameter 162 is substantially equal to the bore diameter 144.

In preferred embodiments, the ratio of the major diameter 122 to the first (or cortical) root diameter 142 can be greater than 1.15:1, such as greater than about 1.2:1, 1.22:1, or 1.24:1, and less than about 1.5:1, 1.45:1, 1.4:1, 1.35:1, or 1.3:1. In preferred embodiments, the ratio of the major diameter 122 to the second (or cancellous) root diameter 162 can be greater than 1.7:1, such as greater than about 1.8:1, 1.9:2:1, or 2.5:1, and less than about 4:1, 3.5:1, or 3:1. In preferred embodiments, the ratio of the first (or cortical) root diameter 142 to the second (or cancellous) root diameter 162 can be greater than 1.5, such as greater than about 1.6:1, 1.8:2, 2:1, 2.2:1, or 2.5:1, and less than about 3.5:1, 3.25:1, 3:1, or 2.75:1.

It will be understood, however, that the suture anchor 100 illustrated in FIGS. 1A-1C can be modified to include any feature(s) shown and/or described in relation to any other suture anchor(s) illustrated in the other Figures described below.

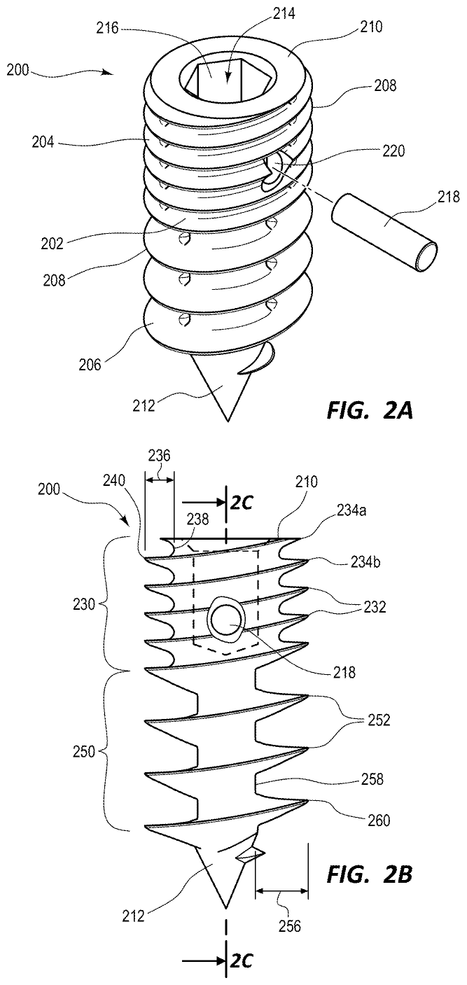

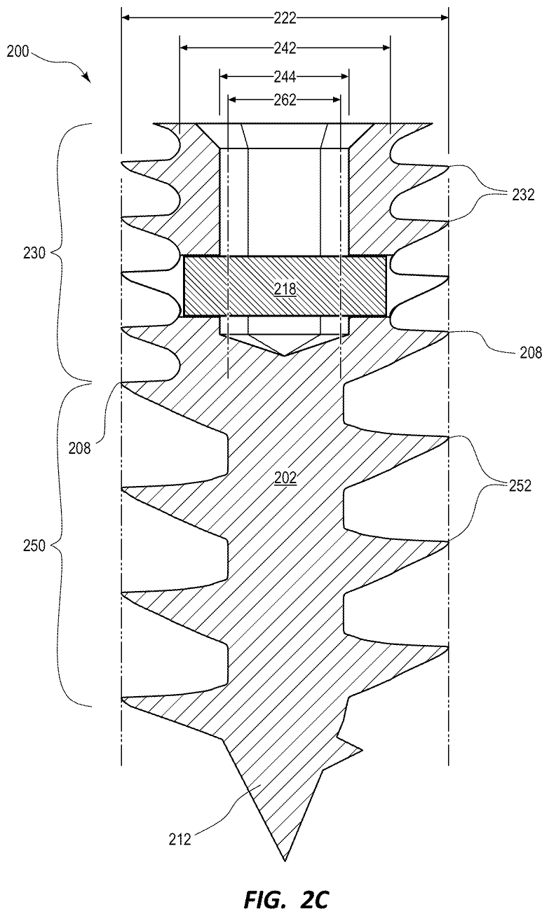

FIGS. 2A-2C illustrate perspective and cross-sectional views of a second embodiment of a suture anchor 200 having an anchor body 202 extending along a longitudinal axis between a proximal end 204 and a distal end 206. FIG. 2C is a cross-sectional view taken along line 2C-2C. The anchor body 202 can have similar proportions and be made of similar material as the anchor body 102 in FIGS. 1A-1C. The 200-series features in FIGS. 2A-2C are illustrated as being substantially the same as their similarly numbered 100-series counterparts illustrated in FIGS. 1A-1C, except as otherwise specified.

One main difference between suture anchor 200 in FIGS. 2A-2C and suture anchor 100 in FIGS. 1A-1C is that the coarse helical threads 252 on anchor body 202 are tapered in the last turn before reaching the distal tip 212. Another main difference is that the second (or cancellous) root diameter 262 is less than the bore diameter 244. It will be understood, however, that the suture anchor 200 can be modified to include any feature(s) shown and/or described in relation to any other suture anchor(s) illustrated in the other Figures.

The anchor body 202 includes highly differentiated thread patterns in a cortical bone-engaging segment 230 and a cancellous bone-engaging segment 250, which optimize engagement with and fixation to, respectively, cortical and cancellous bone regions at the implant site. The cortical bone-engaging segment 230 includes first and second starts 234a and 234b of fine helical threads 232, each preferably having the same pitch as the coarse helical threads 352 in the cancellous bone-engaging segment 350. The main start 234a is a continuation of the coarse helical threads 252 in the cancellous bone-engaging segment 250. In this way, the main start 234a can follow the impression cut by the single start of coarse helical threads 252 as the bone anchor 200 is driven longitudinally into the bone. The secondary start 234b does not follow the impression cut by the single start of coarse helical threads 252 as the bone anchor 200 is driven longitudinally into the bone but creates a new impression between the initial impression cut by the coarse helical threads 252. This further assists in engaging hard cortical bone.

The cortical bone-engaging segment 230 has a first (or cortical) root diameter 242. The central bore 214 has a bore diameter 244. The cancellous bone-engaging segment 250 includes a single start of coarse helical threads 252 having the same pitch as the starts 234 of fine helical threads 232. The ratio of the thread height 256 of the coarse helical threads 252 to the thread height 236 of the fine helical threads 232 can be greater than 1.5:1, such as greater than about 1.6:1, 1.7:1, 1.8:1, or 1.9:1, and less than 4:1, such as less than about 3.8:1, 3.6:1, 3.4:1, 3.2:1 or 3:1

In preferred embodiments, the ratio of the major diameter 222 to the first (or cortical) root diameter 242 can be greater than 1.15:1, such as greater than about 1.2:1, 1.22:1, or 1.24:1, and less than about 1.5:1, 1.45:1, 1.4:1, 1.35:1, or 1.3:1. In preferred embodiments, the ratio of the major diameter 222 to the second (or cancellous) root diameter 262 can be greater than 1.7:1, such as greater than about 1.8:1, 1.9:2:1, or 2.5:1, and less than about 4:1, 3.5:1, or 3:1. In preferred embodiments, the ratio of the first (or cortical) root diameter 242 to the second (or cancellous) root diameter 262 can be greater than 1.5, such as greater than about 1.6:1, 1.8:2, 2:1, 2.2:1, or 2.5:1, and less than about 3.5:1, 3.25:1, 3:1, or 2.75:1.

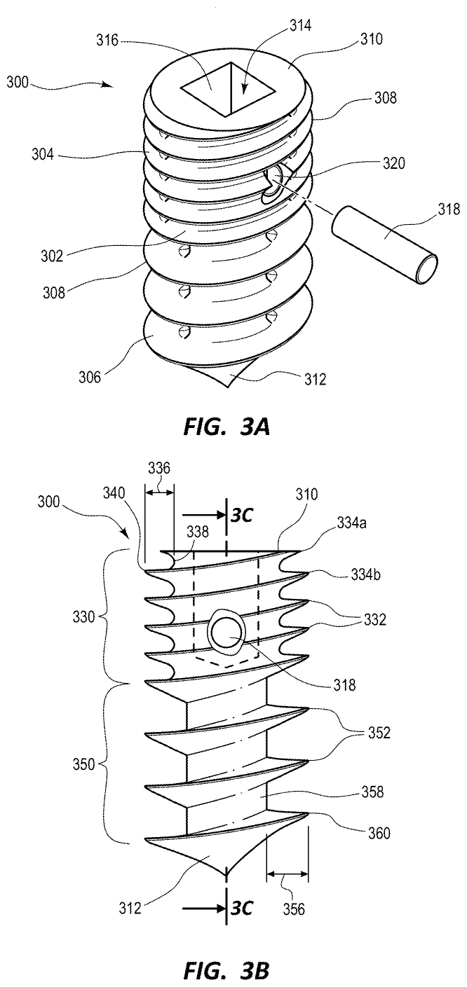

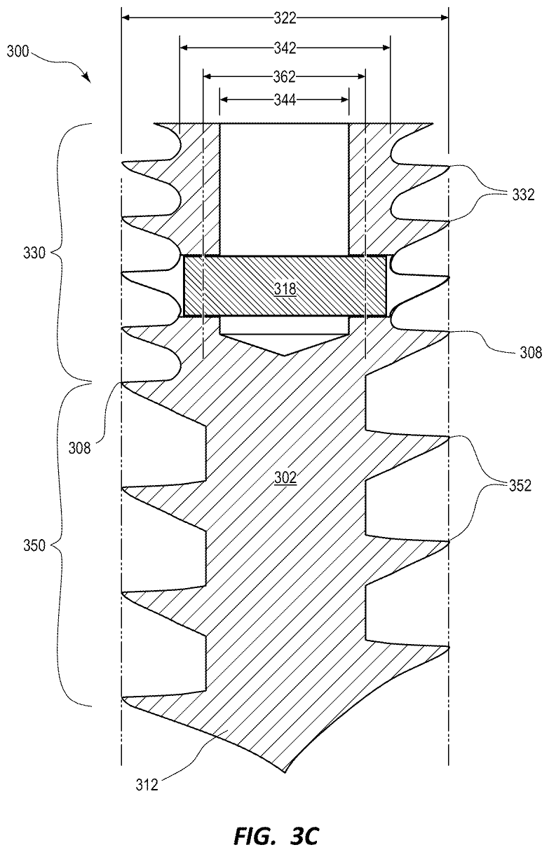

FIGS. 3A-3C illustrate perspective and cross-sectional views of a third embodiment of a suture anchor 300 having an anchor body 302 extending along a longitudinal axis between a proximal end 304 and a distal end 306. FIG. 3C is a cross-sectional view taken along line 3C-3C. The anchor body 302 can have similar proportions and be made of similar materials as the anchor body 102 in FIGS. 1A-1C and/or the anchor body 202 in FIGS. 2A-2C. The 300-series features in FIGS. 3A-3C are illustrated as being substantially the same as their similarly numbered 100-series counterparts illustrated in FIGS. 1A-1C, except as otherwise specified.

One main difference between suture anchor 300 and suture anchor 100 is that the socket 316 is illustrated as square rather than hexagonal. Another main difference is that the second (or cancellous) root diameter 362 is slightly greater than the bore diameter 344. It will be understood, however, that the suture anchor 300 can be modified to include any feature(s) shown and/or described in relation to any other suture anchor(s) illustrated in the other Figures.

The anchor body 302 includes highly differentiated thread patterns in a cortical bone-engaging segment 330 and a cancellous bone-engaging segment 350, which optimize engagement with and fixation to, respectively, cortical and cancellous bone regions at the implant site. The cortical bone-engaging segment 330 includes first and second starts 334a and 334b of fine helical threads 332, each preferably having the same pitch as the coarse helical threads 352 in the cancellous bone-engaging segment 350. The main start 334a is a continuation of the coarse helical threads 352 in the cancellous bone-engaging segment 350. In this way, the main start 334a can follow the impression cut by the single start of coarse helical threads 352 as the bone anchor 300 is driven longitudinally into the bone. The secondary start 334b does not follow the impression cut by the single start of coarse helical threads 352 as the bone anchor 300 is driven longitudinally into the bone but creates a new impression between the initial impression cut by the coarse helical threads 352. This further assists in engaging hard cortical bone.

The cortical bone-engaging segment 330 has a first (or cortical) root diameter 342. The central bore 314 has a bore diameter 344. The cancellous bone-engaging segment 350 includes a single start of coarse helical threads 352 having the same pitch as the starts 334 of fine helical threads 332. The ratio of the thread height 356 of the coarse helical threads 352 to the thread height 336 of the fine helical threads 332 can be greater than 1.5:1, such as greater than about 1.6:1, 1.7:1, 1.8:1, or 1.9:1, and less than 4:1, such as less than about 3.8:1, 3.6:1, 3.4:1, 3.2:1 or 3:1.

In preferred embodiments, the ratio of the major diameter 322 to the first (or cortical) root diameter 342 can be greater than 1.15:1, such as greater than about 1.2:1, 1.22:1, or 1.24:1, and less than about 1.5:1, 1.45:1, 1.4:1, 1.35:1, or 1.3:1. In preferred embodiments, the ratio of the major diameter 322 to the second (or cancellous) root diameter 362 can be greater than 1.7:1, such as greater than about 1.8:1, 1.9:2:1, or 2.5:1, and less than about 4:1, 3.5:1, or 3:1. In preferred embodiments, the ratio of the first (or cortical) root diameter 342 to the second (or cancellous) root diameter 362 can be greater than 1.5, such as greater than about 1.6:1, 1.8:2, 2:1, 2.2:1, or 2.5:1, and less than about 3.5:1, 3.25:1, 3:1, or 2.75:1.

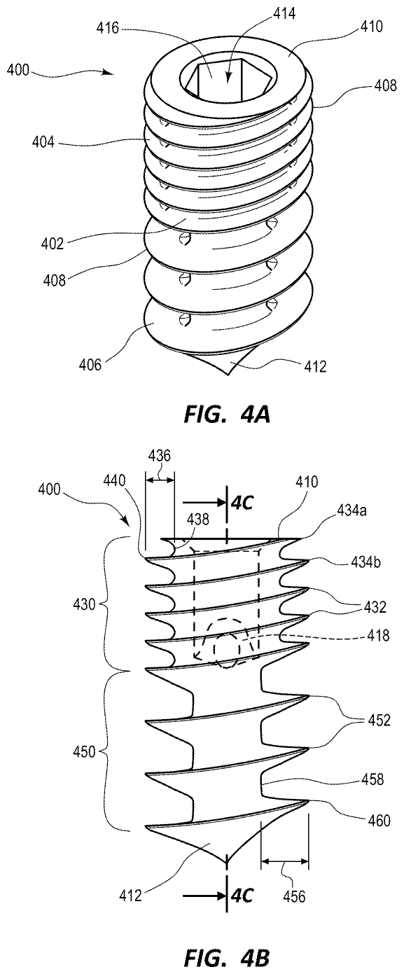

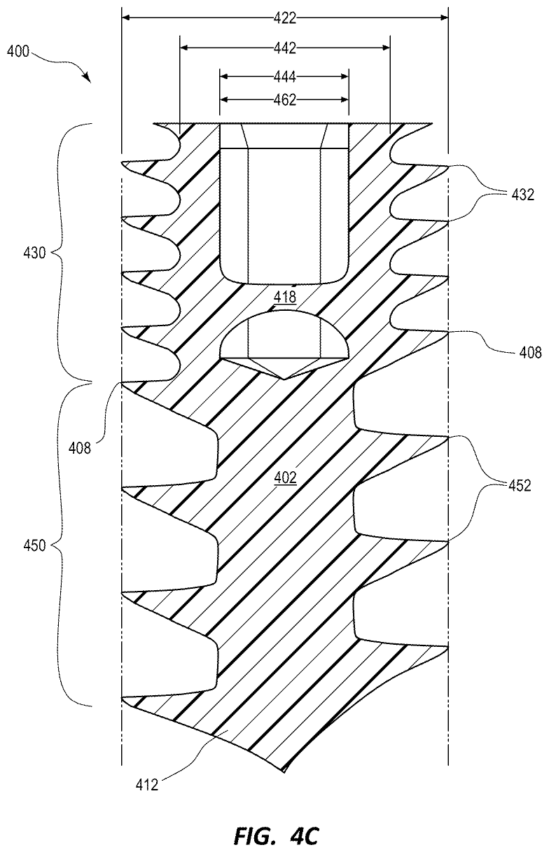

FIGS. 4A-4C illustrate perspective and cross-sectional views of a fourth embodiment of a suture anchor 400 having an anchor body 402 extending along a longitudinal axis between a proximal end 404 and a distal end 406. FIG. 4C is a cross-sectional view taken along line 4C-4C. The anchor body 402 can have similar proportions and be made of similar materials as the anchor body 102 in FIGS. 1A-1C, the anchor body 302 in FIGS. 2A-2C, and/or the anchor body 302 in FIGS. 3A-3C. The 400-series features in FIGS. 4A-4C are illustrated as being substantially the same as their similarly numbered 100-series counterparts illustrated in FIGS. 1A-1C, except as otherwise specified.

An important difference between suture anchor 400 and suture anchor 100 is that the rigid member 418 is illustrated as being integrally formed with the anchor body 402 (such as by one- or two-stage molding, machining, and/or etching), as compared to the rigid pin member 118 being attached through the hole 120 in the sidewall(s) of the anchor body 102. It will be understood, however, that the suture anchor 400 can be modified to include any feature(s) shown and/or described in relation to any other suture anchor(s) illustrated in the other Figures.

The anchor body 402 includes highly differentiated thread patterns in a cortical bone-engaging segment 430 and a cancellous bone-engaging segment 450, which optimize engagement with and fixation to, respectively, cortical and cancellous bone regions at the implant site. The cortical bone-engaging segment 430 includes two starts 434a and 434b of fine helical threads 432. The cortical bone-engaging segment 430 has a first (or cortical) root diameter 442. The central bore 414 has a bore diameter 444. The cancellous bone-engaging segment 450 includes a single start of coarse helical threads 452 having the same pitch as the starts 434 of fine helical threads 432. The ratio of the thread height 456 of the coarse helical threads 452 to the thread height 436 of the fine helical threads 432 can be greater than 1.5:1, such as greater than about 1.6:1, 1.7:1, 1.8:1, or 1.9:1, and less than 4:1, such as less than about 3.8:1, 3.6:1, 3.4:1, 3.2:1 or 3:1.

In preferred embodiments, the ratio of the major diameter 422 to the first (or cortical) root diameter 442 can be greater than 1.15:1, such as greater than about 1.2:1, 1.22:1, or 1.24:1, and less than about 1.5:1, 1.45:1, 1.4:1, 1.35:1, or 1.3:1. In preferred embodiments, the ratio of the major diameter 422 to the second (or cancellous) root diameter 462 can be greater than 1.7:1, such as greater than about 1.8:1, 1.9:2:1, or 2.5:1, and less than about 4:1, 3.5:1, or 3:1. In preferred embodiments, the ratio of the first (or cortical) root diameter 442 to the second (or cancellous) root diameter 462 can be greater than 1.5, such as greater than about 1.6:1, 1.8:2, 2:1, 2.2:1, or 2.5:1, and less than about 3.5:1, 3.25:1, 3:1, or 2.75:1.

FIGS. 5A-5C illustrate a suture anchor assembly for installing a suture anchor into a bone. The suture anchor assembly comprises a suture anchor 500 and a driver 570 comprising a drive shaft 572, a driver tip 574, and a handle (not shown). The driver tip 574 has a size and shape so that it can be inserted into and correspond to the shape of a socket 516 in the suture anchor 500 (FIG. 5A). In use, the driver 570 is connected to the suture anchor 500 and rotated while applying downward pressure to drive the suture anchor 500 into a bone 580 at a surgical site (FIGS. 5B and 5C). The bone 580 has a layer of hard, dense cortical bone 582 at the surface and an underlying region of softer and porous cancellous bone 584. At least the cortical bone layer 582 can be pre-drilled with a pilot hole and/or the suture anchor 500 can be configured to be self-drilling.

One or more sutures 586 can be looped around a rigid member 518 at or near the bottom of a central bore 514 (or directly or indirectly attached to the rigid member 518 in some other fashion). Free ends 588 of the suture(s) 586 can be threaded through a hole in the driver 570, such as through at least a portion of the drive shaft 572. The rigid member 518 provides a suture-attachment site that prevents longitudinal pullout of the sutures 586 and causes an implanted end of the sutures 586 to be fixed at a single location within the bone 580 at a surgical site. The suture anchor 500 includes a cortical bone-engaging segment 530 configured to engage with the cortical bone 582 a cancellous bone-engaging segment 550 configured to engage with the cancellous bone 584. These features maximize the ability of the anchor 500 to fix and retain the implanted end of the sutures 586 at a fixed location within the bone 580.

FIG. 6 illustrates a bone anchor 600 having been installed within a typical bone 680 having a cortical bone layer 682 and a cancellous bone region 684 (such as by using a driver as illustrated in FIGS. 5A-5C). The cortical bone region 682 comprises hard, dense bone, while the cancellous bone region 684 comprises less dense and more porous bone (e.g., that is soft or spongy). The cortical bone-engaging segment 630 is positioned within the cortical bone layer 682 and the cancellous bone-engaging segment 650 is positioned within the cancellous bone region 684.

When the bone anchor 600 is properly inserted into the bone 680, the proximal face 610 of the anchor body 602 will preferably be substantially flush with the surrounding bone surface. This maximizes contact with and mechanical engagement between the cortical bone-engaging segment 630 with its associated fine helical threads 632, and the adjacent cortical bone 682. The coarse helical threads 652 of the cancellous bone-engaging segment 650 have optimized contact with and engagement with the adjacent cancellous bone 684.

The rigid member 618 provides a suture-attachment site that prevents longitudinal pullout of the sutures 686 and causes an implanted end of the sutures 686 to be fixed at a single location within the bone 680 at a surgical site. The specialized cortical and cancellous bone-engaging segments 630, 650 maximize the ability of the anchor 600 to mechanically fix and retain the implanted end of the sutures 686 at a fixed location within the bone 680.

In manufacturing any of the suture anchors disclosed herein, the anchor body and/or rigid member can be cast and formed in a die and/or machined. Alternatively, the anchor body can be cast, formed and/or machined, and the rigid member attached later. For example, the anchor body can be cast and formed from a biodegradable polymer, such as poly-l-lactic acid (PLLA). The anchor body can then be drilled to prepare holes for insertion and fixation of the rigid member across the central bore.

The suture anchors can be distributed to practitioners with one or more sutures threaded through the central bore and attached to the rigid member. An example of a type of suture suitable for use in conjunction with the bone anchor of the present invention is #2 braided polyester. If more than one strand of sutures is used, the sutures can be of a different color and/or color pattern to facilitate identification by the surgeon during a surgical procedure.

FIG. 7 illustrates the use of a suture anchor 700 in a rotator cuff repair surgery. The suture anchor 700 is placed in a humerus bone 780, and sutures 786 are passed through a rotator cuff 790 and tied. Before the suture anchor 700 is inserted in the humerus bone 780, a pilot hole may optionally be drilled. The suture anchor 700 can be inserted into the pilot hole using a driver tool until a proximal face 710 is substantially flush with the surrounding surface of the humerus bone 780. The suture anchor 700 is advantageously placed in the humerus bone 780 at an angle to the tangent of the humerus bone, which is known as the “dead man's angle.”

Because the suture anchor 700 is placed in the humerus bone 780 at an angle, and the suture anchor includes specialized cortical and cancellous bone-engaging segments, the anchor body 702 provides a mechanical advantage against the bone anchor 700 moving laterally and opening the angle to the tangent. By preventing lateral movement, the suture anchor 700 prevents sutures 786 from loosening once they have been properly fastened (e.g., tied) to the rotator cuff 790.

The proximal face 710 of the suture anchor 700 can be substantially flat or non-protruding such that the suture anchor 700 can be placed at or just below the surface of the humerus bone 780. Fine helical threads extend to the proximal face 710 such that the suture anchor 700 has maximum engagement with and mechanical fixation to the humerus bone 780. The opening at the proximal face 710 also allows for the sutures 786 to exit the central bore. The exit opening of the bore can be smooth and curved to enable the sutures 786 to easily slide thereon.

FIG. 8 illustrates the use of multiple suture anchors 800 in a hip repair surgery, such as the repair of a labrum 890 using sutures 886.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A suture anchor for enhanced engagement with and mechanical fixation to cortical and cancellous bone tissues at a surgical site, comprising:
an anchor body extending along a longitudinal axis between a proximal end and a distal end;
helical threads on an outer surface of the anchor body and extending at least partially between a proximal face and a distal tip, the helical threads defining a major diameter of the anchor body;
a central bore that opens at the proximal face and includes a socket for receiving a correspondingly-shaped driver tip; and
a rigid member disposed in the central bore and that provides a suture-attachment site;
the anchor body including a cortical bone-engaging segment beginning at the proximal face and extending partially toward the distal tip and a cancellous bone-engaging segment between the cortical bone-engaging segment and the distal tip,
the cortical bone-engaging segment having a first root diameter and including multiple starts of fine helical threads, the fine helical threads having a thread height,
the cancellous bone-engaging segment having a second root diameter less than the first root diameter and including coarse helical threads having at least one fewer start than the fine helical threads, the coarse helical threads having a thread height greater than the thread height of the fine helical threads,
wherein the central bore extends at least partially through the cortical bone-engaging segment but not into the cancellous bone-engaging segment.

2. The suture anchor of claim 1, further comprising at least one suture attached to around the rigid member.

3. The suture anchor of claim 2, wherein the at least one suture comprises a tissue-securing suture with free ends exiting the central bore and extending beyond the proximal face.

4. The suture anchor of claim 1, wherein the anchor body comprises a material selected from the group consisting of stainless steel, titanium, nickel-titanium alloy, metal, alloys, and polymers.

5. The suture anchor of claim 1, wherein the central bore extends at least partially through the cortical bone-engaging segment but not into the cancellous bone-engaging segment and wherein the rigid member is positioned at a bottom of the central bore proximal to a transition where the cortical bone-engaging segment ends and the cancellous bone-engaging segment begins.

6. The suture anchor of claim 1, wherein the rigid member comprises a transverse pin passing through at least one wall of the anchor body and extending at least partially across the central bore.

7. The suture anchor of claim 1, wherein the rigid member is integrally formed with the anchor body.

8. The suture anchor of claim 1, wherein the socket has a shape selected from hexagonal, pentagonal, square, triangular, star-shaped, oval, or other non-circular or non-cylindrical geometric shape.

9. The suture anchor of claim 1, wherein the anchor body includes a non-threaded portion between the cancellous bone-engaging segment and the distal tip.

10. The suture anchor of claim 1, wherein a ratio of the major diameter to the first root diameter is greater than 1.15:1 and less than about 1.5:1.

11. The suture anchor of claim 1, wherein a ratio of the major diameter to the second root diameter is greater than 1.7:1 and less than 4:1.

12. The suture anchor of claim 1, wherein a ratio of the first root diameter to the second root diameter is greater than 1.6:1 and less than 3.5:1.

13. The suture anchor of claim 1, wherein a ratio of the thread height of the coarse helical threads in the cancellous bone-engaging segment to the thread height of the fine helical threads in the cortical bone-engaging segment is greater than 1.5:1 and less than 3.8:1.

14. A suture anchor system comprising:
the suture anchor of claim 1; and
a driver comparing a driver tip with a size and shape so as to be received within and mechanically engage the socket of the suture anchor.

15. The suture anchor system of claim 14, wherein the driver includes a hole through which a suture, when attached to the suture anchor, can be threaded.

16. A suture anchor for enhanced engagement with and mechanical fixation to cortical and cancellous bone tissues at a surgical site, comprising:
an anchor body extending along a longitudinal axis between a proximal end and a distal end;
helical threads on an outer surface of the anchor body and extending at least partially between a proximal face and a distal tip, the helical threads defining a major diameter of the anchor body;
a central bore that opens at the proximal face and includes a socket for receiving a correspondingly shaped driver tip; and
a rigid member disposed in the central bore and that provides a suture-attachment site;
the anchor body including a cortical bone-engaging segment beginning at the proximal face and extending partially toward the distal tip and a cancellous bone-engaging segment between the cortical bone-engaging segment and the distal tip,
the cortical bone-engaging segment including multiple starts of fine helical threads, the fine helical threads having a thread height defined by a distance between an outer root surface of the anchor body in the cortical bone-engaging segment and outer edges of the fine helical threads,
the cancellous bone-engaging segment including coarse helical threads having at least one fewer start than the fine helical threads, the coarse helical threads having a thread height defined by a distance between an outer root surface of the anchor body in the cancellous bone-engaging segment and outer edges of the coarse helical threads,
wherein the central bore extends through the cortical bone-engaging segment but terminates shy of the cancellous bone-engaging segment,
wherein the rigid member is positioned at or near a bottom of the central bore proximal to where the cortical bone-engaging segment ends and the cancellous bone-engaging segment begins,
wherein the cortical bone-engaging segment has a root diameter greater than a root diameter of the cancellous bone-engaging segment,
wherein the thread height of the coarse helical threads is greater than the thread height of the fine helical threads.

17. The suture anchor of claim 16, further comprising at least one tissue-securing suture looped around the rigid member, wherein the at least one tissue-securing suture has free ends exiting the central bore and extending beyond the proximal face.

18. A suture anchor system comprising:
the suture anchor of claim 16; and
a driver comparing a driver tip with a size and shape so as to be received within and mechanically engage the socket of the suture anchor.

19. A suture anchor for enhanced engagement with and mechanical fixation to cortical and cancellous bone tissues at a surgical site, comprising:
an anchor body extending along a longitudinal axis between a proximal end and a distal end;
helical threads on an outer surface of the anchor body and extending at least partially between a proximal face and a distal tip, the helical threads defining a major diameter of the anchor body;
a central bore that opens at the proximal face and includes a socket for receiving a correspondingly shaped driver tip; and
a rigid member disposed in the central bore and that provides a suture-attachment site;
the anchor body including a cortical bone-engaging segment beginning at the proximal face and extending partially toward the distal tip and a cancellous bone-engaging segment between the cortical bone-engaging segment and the distal tip,
the cortical bone-engaging segment having a first root diameter and including two starts of fine helical threads, the fine helical threads having a thread height,
the cancellous bone-engaging segment having a second root diameter and including coarse helical threads having a single start, the coarse helical threads having a thread height,
wherein the central bore extends at least partially through the cortical bone-engaging segment but not into the cancellous bone-engaging segment,
wherein the rigid member is positioned at or near a bottom of the central bore proximal to where the cortical bone-engaging segment ends and the cancellous bone-engaging segment begins,
wherein the suture anchor is characterized by at least one of:
a ratio of the major diameter to the first root diameter is greater than 1.15:1 and less than about 1.5:1,
a ratio of the major diameter to the second root diameter is greater than 1.7:1 and less than 4:1,
a ratio of the first root diameter to the second root diameter is greater than 1.6:1 and less than 3.5:1, or
a ratio of the thread height of the coarse helical threads to the thread height of the fine helical threads is greater than 1.5:1 and less than 3.8:1.

20. A suture anchor system comprising:
the suture anchor of claim 19; and
a driver comparing a driver tip with a size and shape so as to be received within and mechanically engage the socket of the suture anchor.
